(19) United States
(12) Patent Application Publication
(10) Pub. No.: US 2022/0266169 A1
(43) Pub. Date: Aug. 25, 2022

(54) PRODUCT AGNOSTIC TOOL FOR QUANTIFYING SEPARABILITY AND ORTHOGONALITY FOR INDIVIDUAL AND SEQUENTIAL SEPARATION PROCESSES

(71) Applicant: RENSSELAER POLYTECHNIC INSTITUTE, Troy, NY (US)

(72) Inventors: Steven M. CRAMER, Niskayuna, NY (US); Nicholas Anthony VECCHIARELLO, Troy, NY (US); Camille Louise BILODEAU, Troy, NY (US)

(73) Assignee: RENSSELAER POLYTECHNIC INSTITUTE, Troy, NY (US)

(21) Appl. No.: 17/605,645

(22) PCT Filed: Apr. 23, 2020
(86) PCT No.: PCT/US20/29537

§ 371 Date: Oct. 22, 2021

Related U.S. Application Data
(60) Provisional application No. 62/837,629, filed on Apr. 23, 2019.

(51) Int. Cl. B01D 15/18; C07K 1/18; B01D 15/38; G01N 30/86

(52) U.S. Cl. CPC B01D 15/1878; C07K 1/18; B01D 15/3847; G01N 30/8658

(57) ABSTRACT

This tool utilizes orthogonality concepts used for analytical chromatography and apply them to chromatography for downstream processing applications utilizing a small set of product-agnostic, optimally orthogonal resins with which most separations (capture or polishing) can be accomplished. Libraries of components for separation mediums are provided. The library of components is administered to the separation mediums and combination of the separation mediums at varying pHs, and the separability and orthogonality of each is quantified. The separability is a measure of a probability that the separation mediums will separate a pair of components, whereas the orthogonality is a measure of the enhancement in separability upon addition of another separation medium. By identifying those combinations of separation mediums that not only provide advantageous separability, but also high orthogonality, sets of separation mediums can be more easily provided or wholly designed for use in processing applications.

### CROSS REFERENCE TO RELATED APPLICATION(S)

This application is a national stage filing of International Application No. PCT/US2020/029537, filed on Apr. 23, 2020, which claims the benefit of U.S. Provisional Application No. 62/837,629, filed Apr. 23, 2019, the disclosures of which are incorporated by reference as if disclosed herein in their entireties.

### BACKGROUND

Currently, non-affinity purification challenges are addressed using a wide range of resins that can be operated under a variety of conditions resulting in a large process design space. While this large design space may encompass a process that can solve a given separation, it is often challenging, and even untenable, to fully explore this space.

The extent of an orthogonal separation in downstream purification is a concept that many practicing chromatographers intuitively and heuristically understand yet could likely not quantify. When asked to provide an example of two orthogonal resins, one might say that an ion exchange chromatography (IEX) and hydrophobic interaction chromatography (HIC) are orthogonal modes of separation. Yet, even within these two categories of chromatography, there is diversity of ligand chemistry which may give rise to differences in selectivity resulting in more or less orthogonality. For example, does a HIC resin with a phenyl group provide better orthogonality to a cation exchanger than a HIC resin with an alkyl group does? To further complicate this, other modes of chromatography are far less intuitive with regard to their extent of orthogonality. In particular, extent of orthogonality in multimodal separations can be particularly challenging to intuitively know due to the complexity and synergy of different modes in interactions. For example, the arrangement of charge group and hydrophobic groups between two multimodal cation exchangers may be very different and allow each resin to interrogate very different patches on a protein surface. As a result, these two multimodal cation exchangers may have very different selectivity trends and be highly orthogonal, despite the similarity in functionality. Understanding and quantifying these nonintuitive orthogonality trends could be invaluable not only from a process development and resin selection perspective, but also from a resin design and even fundamental molecular interaction perspective.

Many different methods exist for quantifying the extent of orthogonality in analytical chromatography systems, e.g., the Geometric Surface Coverage (SCG), the Gilar-Stoll Surface Coverage (SCS), and the Box Counting Dimensionality (DBC) methods. These methods can be classified as being discrete-based methods since they rely on partitioning the separation space into discrete bins. Other discrete methods also exist which are based on information theory, such as the Mutual Information Method, however, these methods do not rely on graphical based approaches.

FIGS. 1A and 1B provide graphical examples of both of these methods. The SC_{G} method (FIG. 1A) involves creating a scatter plot with one axis corresponding to the retention times of all proteins on column 1 and the other axis corresponding to the retention times of all proteins on column 2. The scatter plot is then divided into B bins of equal size. The fractional surface coverage is then determined by dividing the total number of bins which contain at least one point by the quantity B. In the example in FIG. 1A, the SC_{G} value is 0.28 since 7 bins (shaded) contain points out of a total of 25 bins. Notice that the number of points, or proteins (N) equals the number of bins, such that a completely orthogonal system will have one point in each bin (SC_{G}=1) while a completely non-orthogonal system will have all points in a single bind (SC_{G}=1/B). It has been shown that this method is only robust for the case when the number of bins equals the number of proteins (N=B). When this constraint does not hold true, the maximum possible value for an optimally orthogonal system is N/B. In addition, when N≠B, SC_{G} values can only be compared between datasets that use the same number of proteins and bins. For example, if we are comparing all possible pair combinations from a set of M chromatograms, where M>2, to determine which pairs are the most orthogonal, then all chromatograms have the same number of proteins and all pairwise comparisons use the same number of bins.

Another similar method is the Gilar-Stoll Surface Coverage (SCs) method for calculating orthogonality and this approach is illustrated in FIG. 1B. Similar to the SC_{G} method, bins containing at least one point are shaded in gray. Instead of determining the fraction of gray boxes, a region is segmented which surrounds the perimeter of the grey boxes such that all boxes are contained in this space and that the perimeter can only contain corners of 90°. The SCs value is then calculated by dividing the total number of boxes contained in this region by the total number of boxes. In the example in FIG. 1B, the region outlined in red contains 8 boxes out of a total of 25 boxes, so SC_{S}=0.32. One benefit of this method is that it has been shown to be less sensitive to protein number and box size than the SC_{G} method. The drawback to the SCs method is that is incorporates boxes which do not contain points, and as a result, overestimates orthogonality in systems with outlier points.

Another widely used, discrete method for calculating orthogonality in the analytical chromatography space is the Box Counting Dimensionality (DB_{C}) method. This method attempts to calculate the fractal dimension of the spread of the data when a scatter plot is made using the retention times of a set of proteins on 2 chromatograms (such as the scatter plots shown in FIGS. 1A-1B). Higher fractal dimensions correspond to more “space filling” in these scatter plots, and according to this definition, indicates a greater degree of orthogonality. To calculate orthogonality using this method, the number of bins (N) that the scatter plot is segmented into is varied over a range. For each value of N, the number of occupied bins is determined. A plot of the log of the bin (or box) size vs. log of number of bins (or boxes) occupied is created and an example of this is shown in FIG. 2. Black dots represent data generated from random data using 100 points. The red line shows the best fit line in the linear region, forced through the origin. The fit used the first 10 data points (from right to left) since inclusion of more data points resulted in a fit with an R² value <0.99. The slope of this line is shown, and the DB_{C}=−Slope. For this example, two random datasets (one for each resin) of 100 proteins retentions normalized from 0 to 1 were generated in Matlab. To calculate the DB_{C} (not to be confused with the dynamic binding capacity), a line (red) going through the origin is fit to the linear region of the plot. Points are included in the fit from right to left until the R² value drops below some threshold (R²≥0.99 for this example). The slope of this line is equal to −DB_{C}. It is important to note that this approach empirically still works even if the data does not contain fractal properties since this approach indirectly calculates the extent of space filling.

The other types of method commonly used to calculate orthogonality are non-discrete methods, meaning that they do not divide the separation space into boxes or bins. The three types of methods are correlation coefficient methods, the convex hull method, the nearest neighbor methods.

The first method involves correlation coefficients. Several different types of correlations coefficients have been used, including the Pearson, Spearman, and Kendall coefficients, in an attempt to calculate orthogonality in chromatographic systems. These methods for calculating orthogonality in chromatographic systems have been criticized, however, for several reasons. Firstly, correlations coefficients are better at quantifying the degree of correlation between data rather than quantifying how uncorrelated two data sets are. In addition, it has been shown that correlation coefficients poorly perform when datasets exhibit outliers or clusters of points. Finally, the extent of orthogonality is highly dependent on the system size as well as the selection of data. This was shown by randomly sampling N points from a dataset of 196 proteins and measuring the RMSD between 8 samplings. Correlation coefficients showed the highest RMSD between all orthogonality metrics which were considered in this study.

Another non-discrete method that we will discuss is the Convex Hull Method (SCCH) for quantifying orthogonality. In this method, a scatter plot is again created for the set of protein retention times for a pair of resins/conditions and a convex hull region is created. This region is the smallest possible (by area) which contains all points in the scatter plot and can be thought of as pulling a rubber band around the set of points. The SCCH value is then calculated by dividing the area contained in this region by the total area. Higher values of SCCH correspond to greater extents of orthogonality since a larger portion of the separation space is contained in this region. FIG. 3 shows an example of this calculation using two real retention datasets of the 15 model proteins on Nuvia cPrime and ToyoPearl MX-Trp-650M, both using pH 5.0 linear salt gradients. The convex hull containing the retention data for the 15 proteins is outlined in red and filled with gray. The gray dotted line in this figure outlines the total possible separation space, which in this case has an area equal to 1. The value for SCCH for this resin/condition pair was found to be 0.1397. The major drawback to this method is that it is very sensitive to outliers, which may artificially increase the area of this convex hull. In the extreme case, for example, where all of the points lie on the corners of the separation space box, this would correspond to an SCCH value of 1.0, which would indicate perfect orthogonality. Although this would indicate that the retention times of the proteins were very different for the two resins/conditions, it would also indicate that many of the proteins would not be separable from each other and would coelute in the flowthrough or strip fractions.

The last method discussed to quantify orthogonality in chromatographic systems uses a nearest neighbor approach, as shown in FIG. 4. Model protein retention data from pH 5.0 salt gradients on Nuvia cPrime (Column 1) and Toyopearl MX-Trp-650M (Column 2) was used in this example. Black dots represent the retention times of individual model proteins. Red lines connect each dot to its nearest neighbor. The data shown in FIG. 4 was generated using the retention times of 15 model proteins in pH 5.0 linear salt gradients on Nuvia cPrime (Column 1) and Toyopearl MX-Trp-650M (Column 2), and is the same data used in FIG. 3. The concept of using a nearest neighbor approach is relatively intuitive and straightforward. For each protein (black points in FIG. 4), the Euclidean distance between itself and every other point is calculated and its nearest neighbor, or the point closest in space, is identified. For clarity, red lines have been drawn between each point's nearest neighbor in FIG. 4. The goal of the nearest neighbor approach is to identify resin/condition sets which exhibit a large spread in the points, or a large difference in selectivity. As a result, a highly orthogonal system is one in which each point has a large nearest neighbor distance. This approach can be particularly useful for calculating orthogonality between resin sets of arbitrary size since nearest neighbor calculations are relatively straightforward and can be quickly calculated in N-dimensional space. Several groups have used nearest neighbors to quantify orthogonality in chromatograms. Common methods include summing all of the nearest neighbor distance, summing the square of the nearest neighbor distances, calculating the arithmetic mean, calculating the harmonic means, or calculating the geometric mean.

The above methods have all been developed for use in the analytical chromatography space in order to identify efficient pairs of chromatography resins to improve peak capacities for complex mixtures of peptide digests. A quantitative method for calculating orthogonality has not been applied for separations in the downstream chromatography space.

### SUMMARY

Some embodiments of the present disclosure are directed to a method for determining an optimized separation including preparing a library of components for separation; preparing a library of separation mediums; administering the library of components to the separation mediums from the library of separation mediums and a plurality of combinations of the separation mediums to separate pairs of components in the library of components; quantifying a separability (S) of the separation mediums and the combinations of separation mediums, wherein the separability (S) is a measure of a probability that the separation medium or combination of separation mediums will separate a pair of components in the library of components; and quantifying an orthogonality (E_{M}) of the combinations of separation mediums, wherein the orthogonality (E_{M}) is a measure of enhancement in separability (S) of a combination of M number of separation mediums over M−1 number of separation mediums. In some embodiments, the method includes identifying properties of the combination of M number of separation mediums and the library of components that increase orthogonality (E_{M}) of a set of separation mediums; identifying a target sample for separation; selecting a set of substantially orthogonal separation mediums for use in separating components of the target sample; and administering the target sample to the set of substantially orthogonal separation mediums. In some embodiments, administering the library of components includes administering the library of components to the separation mediums from the library of separation mediums and the plurality of combinations of separation mediums at varying pH.

In some embodiments, the separability (S) is calculated according to the following Formula I:

$$S = \frac{1}{( \begin{matrix}n \\ 2\end{matrix} )} {\sum }_{a = 1}^{n - 1} {\sum }_{ba = a + 1}^{n} {w}_{a b} \qquad ( Formula I )$$

wherein

$${w}_{ab} = \{ \begin{matrix}0 , & {d}_{ab} < {r}_{low} \\ \frac{{d}_{ab} - {r}_{low}}{{r}_{high} - {r}_{low}} , & {r}_{high} > {d}_{ab} > {r}_{low} \\ 1 , & {d}_{ab} > {r}_{high}\end{matrix} \} ,$$

and
wherein n is the number of components in the library, d_{ab} is the separation distance between a first component (a) and a second component (b) on a separation medium, r_{high} represents the threshold above which first component (a) and second component (b) are considered successfully separated and r_{low} represents the threshold below which first component (a) and second component (b) are considered unsuccessfully separated.

In some embodiments, the orthogonality (E_{M}) is calculated according to the following Formula II:

$$( Formula II )$$

$${E}_{M} = \frac{{S}_{M}}{max ( {S}_{M - 1} ∀ resin / condition combinations in M )} - 1$$

In some embodiments, the separability (S) of the set of substantially orthogonal separation mediums is greater than about 0.5. In some embodiments, the separability (S) of the set of substantially orthogonal separation mediums is greater than about 0.75. In some embodiments, the orthogonality (E_{M}) of the set of substantially orthogonal separation mediums is greater than about 0.2. In some embodiments, the orthogonality (E_{M}) of the set of substantially orthogonal separation mediums is greater than about 0.35.

Some embodiments of the present disclosure are directed to a method for performing an optimized separation. In some embodiments, the method includes providing a sample including a plurality of components, the plurality of components including one or more target components and one or more impurities; identifying properties of the one or more target components and the one or more impurities; selecting a set of M number of substantially orthogonal separation mediums for use in separating the one or more target components from the one or more impurities based on the identified properties, wherein an orthogonality (E_{M}) of the set of substantially orthogonal separation mediums is a measure of enhancement in separability (S) of the M number of separation mediums over M−1 number of separation mediums; and administering the sample to the set of M number of substantially orthogonal separation mediums. In some embodiments, the separability (S) of the set of M number of substantially orthogonal separation mediums is greater than about 0.5. In some embodiments, the separability (S) of the set of M number of substantially orthogonal separation mediums is greater than about 0.75. In some embodiments, the orthogonality (E_{M}) of the set of M number of substantially orthogonal separation mediums is greater than about 0.2. In some embodiments, the orthogonality (E_{M}) of the set of M number of substantially orthogonal separation mediums is greater than about 0.35. In some embodiments, selecting the set of M number of substantially orthogonal separation mediums includes designing a combination of substantially orthogonal separation mediums for the one or more target components and the one or more impurities based on the identified properties.

Some embodiments of the present disclosure are directed to a method for performing an optimized separation. In some embodiments, the method includes preparing a library of proteins for separation; preparing a library of chromatographic separation resins; first administering the library of proteins to the chromatographic separation resins from the library of chromatographic separation resins and a plurality of combinations of the chromatographic separation resins, wherein the chromatographic separation resins and the plurality of combinations of the chromatographic separation resins are maintained at a first pH; subsequently administering the library of proteins to the chromatographic separation resins and the plurality of combinations of the chromatographic separation resins, wherein the chromatographic separation resins and the plurality of combinations of the chromatographic separation resins are maintained at at least a second pH; quantifying a separability (S) of the chromatographic separation resins and the plurality of combinations of the chromatographic separation resins, wherein the separability (S) is a measure of a probability that the chromatographic separation resin or the combination of chromatographic separation resins will separate a pair of proteins in the library of proteins; quantifying an orthogonality (E_{M}) of the combinations of chromatographic separation resins, wherein the orthogonality (E_{M}) is a measure of the enhancement in separability (S) of a combination of M number of chromatographic separation resins over M−1 number of chromatographic separation resins; identifying properties of the combinations of M number of chromatographic separation resins and the library of proteins that increase orthogonality (E_{M}) for a set of chromatographic separation resins; providing a sample including two or more proteins; selecting a set of substantially orthogonal chromatographic separation resins for use in separating the two or more proteins based on the identified properties; and administering the sample to the set of substantially orthogonal chromatographic separation resins. In some embodiments, the separability (S) of the set of substantially orthogonal separation mediums is greater than about 0.75, and the orthogonality (E_{M}) of the set of substantially orthogonal separation mediums is greater than about 0.35.

### BRIEF DESCRIPTION OF THE DRAWINGS

The drawings show embodiments of the disclosed subject matter for the purpose of illustrating the invention. However, it should be understood that the present application is not limited to the precise arrangements and instrumentalities shown in the drawings, wherein:

FIG. 1A is a prior art graphical method for calculating orthogonality of a combination of separation mediums;

FIG. 1B is a prior art graphical method for calculating orthogonality of a combination of separation mediums;

FIG. 2 is a is a prior art graphical method for calculating orthogonality of a combination of separation mediums;

FIG. 3 is a prior art graphical method for calculating orthogonality of a combination of separation mediums;

FIG. 4 is a prior art graphical method for calculating orthogonality of a combination of separation mediums;

FIG. 5 is a chart of a method for determining an optimized separation according to some embodiments of the present disclosure;

FIGS. 6A-6C portray graphs of an exemplary separation analysis according to some embodiments of the present disclosure;

FIGS. 7A-7B portray graphs of an exemplary separation analysis according to some embodiments of the present disclosure;

FIG. 8 is a chart of a method for performing an optimized separation according to some embodiments of the present disclosure;

FIG. 9 is a chart of a method for performing an optimized separation according to some embodiments of the present disclosure;

FIGS. 10A-10C portray chromatograms for model proteins utilized according to some embodiments of the present disclosure;

FIGS. 11A-11B portray chromatograms for model proteins utilized according to some embodiments of the present disclosure;

FIGS. 12A-12D portray elution distributions of model proteins utilized according to some embodiments of the present disclosure; and

FIGS. 13A-13D portray elution distributions of model proteins utilized according to some embodiments of the present disclosure.

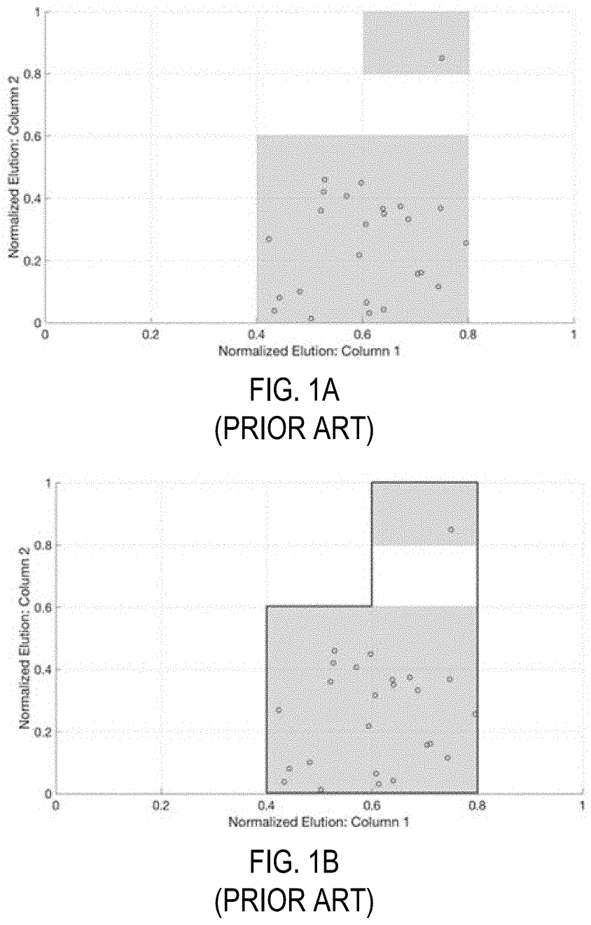

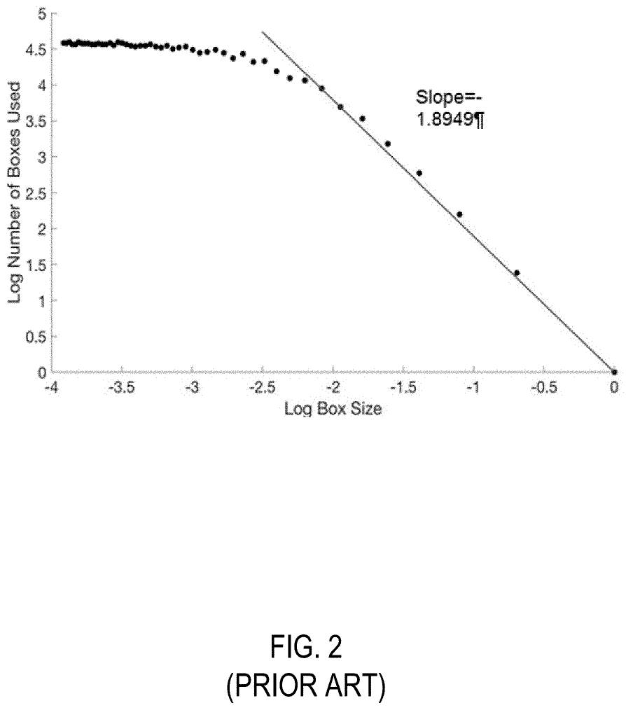

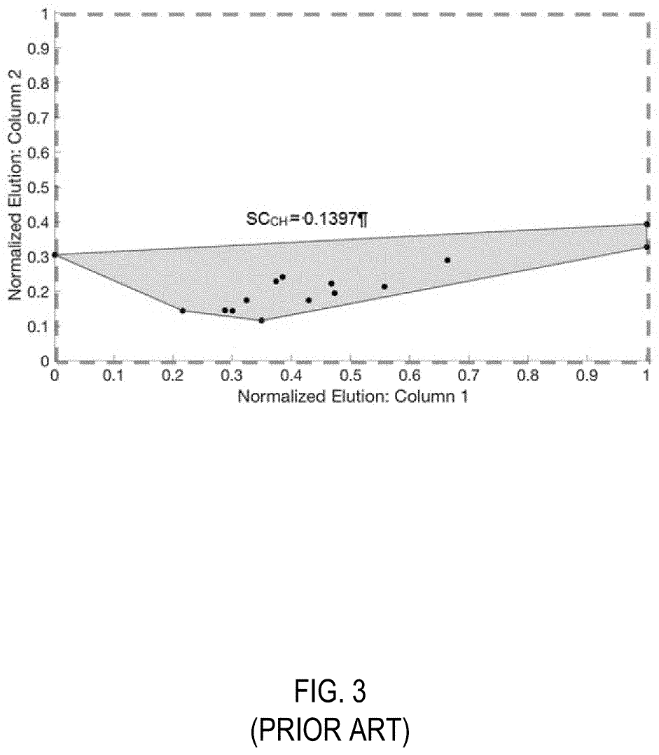

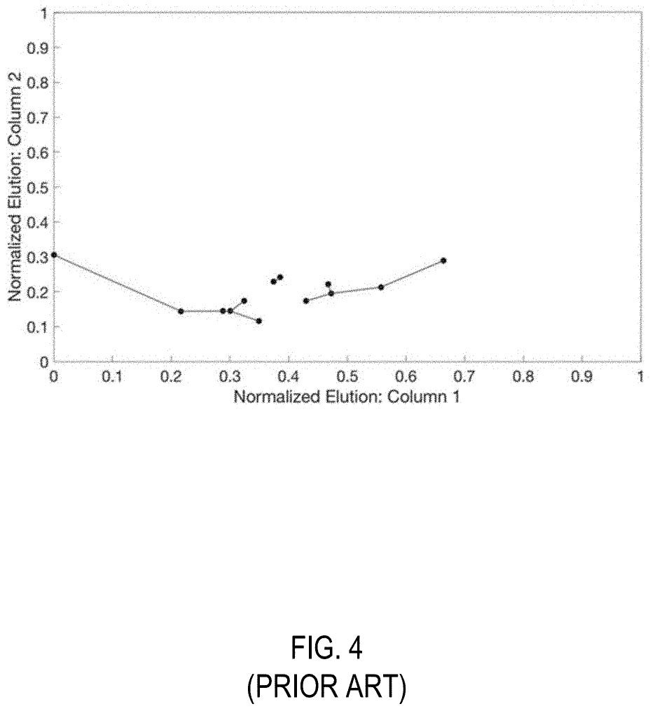

### DETAILED DESCRIPTION

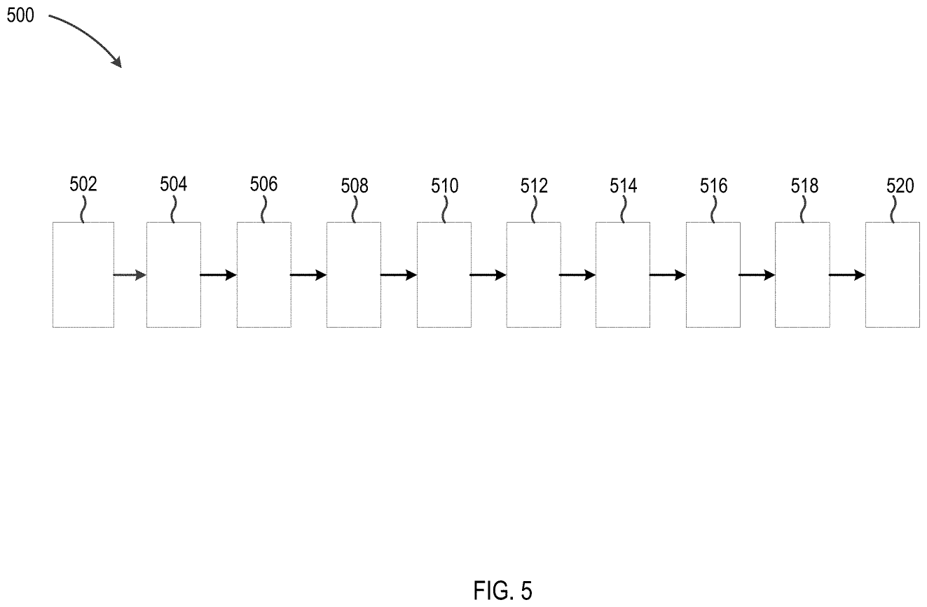

Referring now to FIG. 5, some embodiments of the present disclosure are directed to a method 500 for determining an optimized separation. In some embodiments, method 500 is a graphical-based approach that quantifies separability and orthogonality of separation mediums for use in downstream processes. In some embodiments, the present disclosure is directed to a mathematical framework for determining product-agnostic orthogonality. As used herein, separability is defined as the ability of an individual separation medium or combination of separation mediums to separate components in a sample from each other. Orthogonality, on the other hand, is defined as the enhancement of separability when an additional separation medium is added to separation medium or a combination of separation mediums. The present disclosure refers to “combinations” and “sets” of separation mediums, which are intended to mean two or more separation mediums. In some embodiments of the present disclosure, the separation mediums are any material or process suitable to isolate target components in the sample from impurities in the sample. In some embodiments, the separation mediums include one or more chromatographic separation resins, as will be discussed in greater detail below. In some embodiments, the chromatographic separation resins are configured for use gradient elution chromatography processes. In some embodiments, the target components are any desired component of the sample, e.g., one or more proteins, polymers, small molecules, etc. In some embodiments, the impurities are any components of the sample who presence is undesired, e.g., because they adversely affect the intended function of the sample, target components, etc. In some embodiments, the impurities includes undesired proteins, polymers, small molecules, etc. As will be discussed in greater detail below, in some embodiments, separability and orthogonality are quantified in systems containing arbitrary numbers of components for separation medium sets of arbitrary size.

Still referring to FIG. 5, some embodiments of method 500 include preparing 502 a library of components for separation. In some embodiments, the library includes a plurality of target components, a plurality of impurities, or combinations thereof. In some embodiments, all or substantially all of the components in the library are reference components, i.e., the identity of the components is known. In some embodiments, the library includes a plurality of proteins. Any suitable proteins can be utilized in the library of components, as will be discussed in greater detail below. In some embodiments, the library of components includes catalase, trypsin, alpha-lactalbumin, alpha-chymotrypsinogen A (type II), concanavalin A (type VI), lysozyme, horse cytochrome C, alpha-casein, alpha-chymotrypsin, apoferritin, lipoxidase, ribonuclease B, conalbumin, lipase, ubiquitin, carbonic anhydrase, hemoglobin, lactoferrin, hgh, mab 2, phosphatase (alkaline), beta-lactoglobulin B, alpha amylase (type VIII-A), glargine, albumin (rabbit), etc. In some embodiments, the library of components includes more than 10 components, more than 15 components, more than 20 components, more than 30 components, more than 40 components, more than 50 components, etc.

At 504, a library of separation mediums is prepared. As discussed above, in some embodiments, the separation mediums in the library of separation mediums include one or more chromatographic separation resins. In some embodiments, the chromatographic separation resins are configured for use gradient elution chromatography processes. In some embodiments, the separation mediums include SP Sepharose HP, HyperCel STAR CEX, Capto MMC, Capto MMC ImpRes, Nuvia cPrime, CMM HyperCel, Toyopearl MX-Trp-650M, Eshmuno HCX, BakerBond Poly ABx, Q Sepharose HP, BakerBond PolyQuat, Eshmuno Q, HyperCel STAR AX, Capto Adhere, etc. At 506, the library of components are administered to the separation mediums from the library of separation mediums. At 508, the library of components are administered to a plurality of combinations of the separation mediums. Upon interaction of the library of components with the separation mediums (step 506) and/or the combinations of separation mediums (step 508), pairs of components in the library of components are separated. As discussed above, in some embodiments, the library of components are separated on separation mediums (step 506) and/or combinations of separation mediums (step 508) via gradient elution chromatography processes. In some embodiments, steps 506 and 508 are performed multiple times across a variety of separation mediums. In some embodiments, steps 506 and 508 occur multiple times on the same separation mediums (step 506) and/or combinations of separation mediums (step 508) at a variety of pHs. In some embodiments, the library of proteins is first administered to the chromatographic separation resins and the plurality of combinations of the chromatographic separation resins at a first pH, and then subsequently administered to the chromatographic separation resins and the plurality of combinations of the chromatographic separation resins at least a second pH, third pH, fourth pH, etc.

At 510, a separability (S) of the separation mediums and the combinations of separation mediums is quantified. In some embodiments, separability (S) is a measure of a probability that the separation medium or combination of separation mediums will separate a pair of components in the library of components. In some embodiments, separability (S) is calculated according to the following Formula I:

$$S = \frac{1}{( \begin{matrix}n \\ 2\end{matrix} )} {\sum }_{a = 1}^{n - 1} {\sum }_{b = a + 1}^{n} {w}_{ab} \qquad ( Formula I )$$

wherein

$${w}_{ab} = \{ \begin{matrix}0 , & {d}_{ab} < {r}_{low} \\ \frac{{d}_{ab} - {r}_{low}}{{r}_{high} - {r}_{low}} , & {r}_{high} > {d}_{ab} > {r}_{low} \\ 1 , & {d}_{ab} > {r}_{high}\end{matrix} \} ,$$

and
wherein n is the number of components in the library, d_{ab} is the separation distance between a first component (a) and a second component (b) on a separation medium, r_{high} represents the threshold above which first component (a) and second component (b) are considered successfully separated and r_{low} represents the threshold below which first component (a) and second component (b) are considered unsuccessfully separated.

At 512, an orthogonality (E_{M}) of the combinations of separation mediums is quantified. As discussed above, orthogonality (E_{M}) is a measure of enhancement in separability (S) when an additional separation medium is added to a combination of separation mediums. In other words, orthogonality (E_{M}) is a measure of enhancement in separability (S) of M number of separation mediums over M−1 number of separation mediums. In some embodiments, orthogonality (E_{M}) is calculated according to the following Formula II:

$${E}_{M} = \frac{{S}_{M}}{max ( {S}_{M - 1} ∀ resin / condition combinations in M )} - 1 \qquad ( Formula II )$$

In this way, the enhancement factor associated with resin/condition combination set M describes the orthogonality within that set, such that a higher enhancement indicates greater orthogonality within the set, while a lower enhancement indicates greater redundancy within the set, i.e., resin/condition combinations in the set tend to separate the same pairs of proteins.

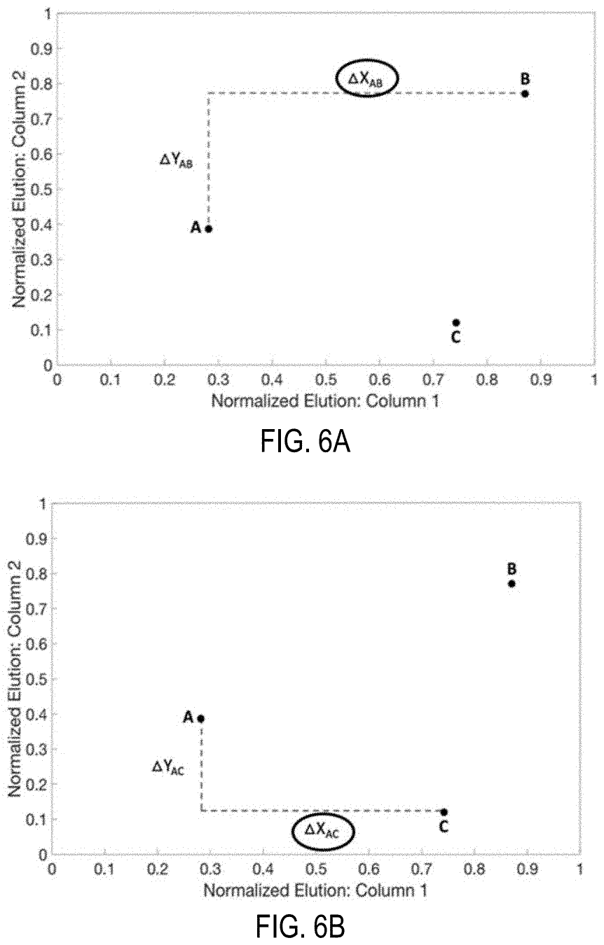

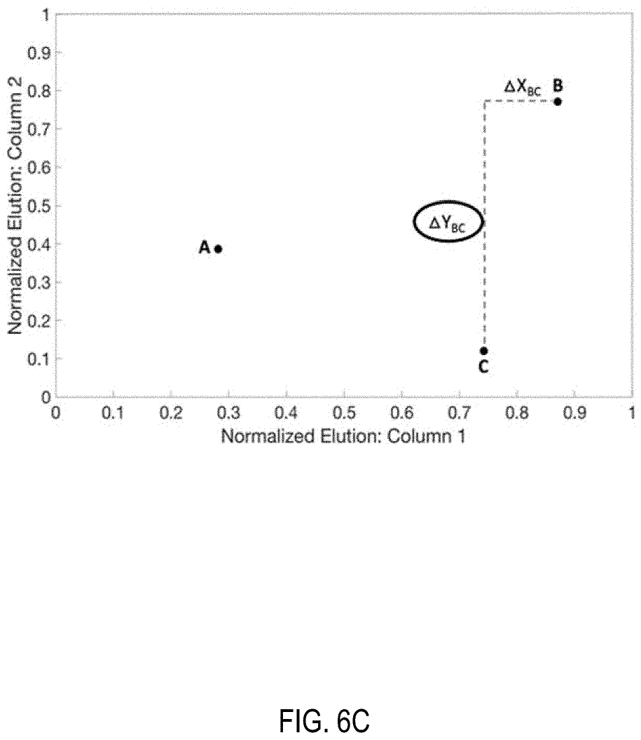

In some embodiments, the above-identified definitions of separability and orthogonality enable a graphical based approach for downstream purification purposes where a set of retention data for m proteins is plotted against n axes, where n is the number of resins/conditions being considered/compared. Referring now to FIGS. 6A-6C, a simple case is portrayed where m=3 and n=2, exemplary of distance calculations for a pair of resins/conditions using randomly generated data in Matlab. Points A, B, and C represent protein retention times in this 2-dimensional scatter plot. For each pair of point, the ΔX_{AB} and ΔY_{AB} are calculated. a.) shows the distances between points A and B, b.) shows the distances between points A and C, and c.) shows the distances between points B and C. The circled quantities show which of the two distances, ΔX_{AB} and ΔY_{AB}, are maximal for each pair of points.

The first step is to calculate the distances between all possible pairs of points, resulting in a total of

$$\frac{2 !}{m ! * ( m - 2 ) !}$$

pairs. Unlike the nearest neighbor methods, which calculate the Euclidean distances between pairs, this method calculates the distances between pairs along each of the n axes, resulting in a total of

$$n * \frac{2 !}{m ! * ( m - 2 ) !}$$

distances. For each pair, the maximum axial distance is found and used in the calculations for separability and orthogonality. For the example in FIGS. 6A-6C, the maximum axial distance for each pair is circled; AX for points A and B (FIG. 6A), ΔX for points A and C (FIG. 6B), and ΔY for points B and C (FIG. 6C). Without wishing to be bound by theory, the use of the maximum axial distance instead of the Euclidean distance minimizes the effect of redundancy. For example, if one dimension is able to provide baseline separation between two proteins, a second dimension which can also baseline separate these two proteins is unnecessary. Euclidean methods would give higher scores for these scenarios, and as a result may not necessarily be useful. In addition, the Euclidean distance is a somewhat arbitrary definition of distance in this case since its distance is not a linear function of the axial distance.

In some embodiments, the term r_{c} (r_{high}) is introduced in order to consider the extent of utilization of relevant separation space in the orthogonality calculations. It sets an upper bound for the separation of two peaks, beyond which additional separation is no longer relevant. For example, if on Resin 1, protein A elutes at 100 mM NaCl and protein B elutes at 850 mM NaCl in a linear salt gradient from 0 to 1500 mM NaCl, there is a 750 mM difference in elution salt concentration. If these two peaks are baseline separated after 300 mM NaCl elution salt difference, additional separation beyond 300 mM NaCl difference has little additional benefit from a practical perspective. In other words, the r_{c} value defines this separation distance at which additional separation is immaterial. In some embodiments, r_{c} is any value between 0 and 1, which corresponds to the cutoff expression as “fraction of the total gradient”. In the example above, if 300 mM NaCl is chosen as the cutoff in the 1500 mM NaCl gradient, this would correspond to a r_{c} value of 0.2. Thus, In some embodiments, when calculating the axial distances, if the maximum axial distance exceeds this r_{c} value, then that distance is instead set to r_{c}. Since r_{c} is tunable, setting its value to 1.0 is mathematically equivalent to eliminating its use in orthogonality calculations.

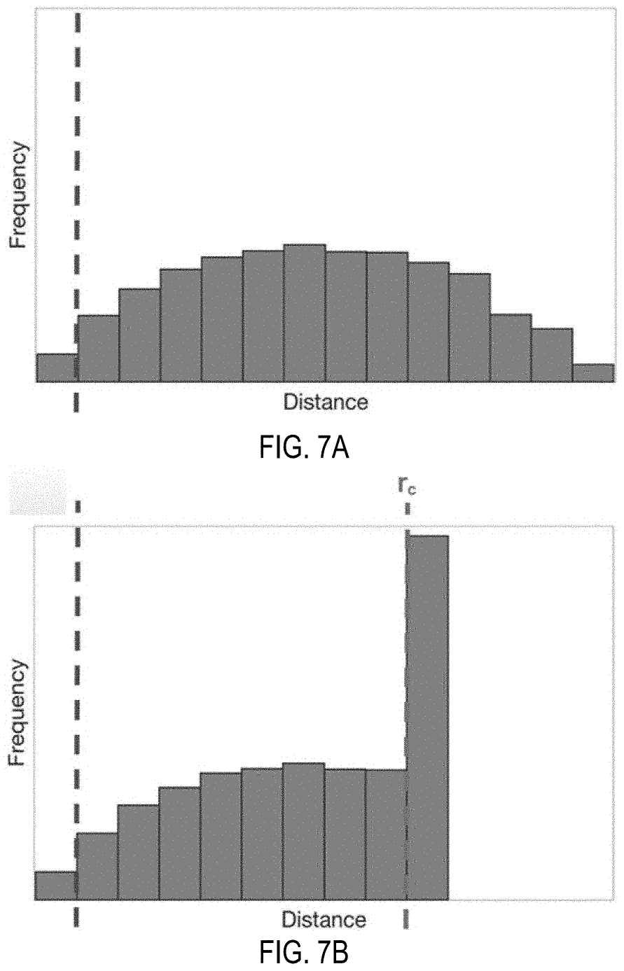

Referring now to FIGS. 7A-7B, in some embodiments, upon calculating the

$$\frac{2 !}{m ! * ( m - 2 ) !}$$

maximal axial distances between all possible protein pairs in a resin set, these distances are then expressed as a distribution, which is related to the probably that a given resin set can separate any two proteins. FIGS. 7A-7B shows an example of how this distribution looks for a set of 100 randomly generated protein retention times on a pair of resins. Example of distance distributions from a set of 100 randomly generated protein retention times on a pair of resins. The distances between protein pairs to the left of the first dotted line indicate proteins which are unlikely to be separable from each other by any appreciable amount, whereas the right dotted line represents the r_{c} (r_{high}) cutoff. FIG. 7A shows the distribution without the r_{c} value (or with r_{c}=1), and FIG. 7B shows how the inclusion of r_{c} affects the distance distribution. Although the example in FIG. 7A shows a distribution which looks Gaussian, the present disclosure is not limited to such samples. As can be seen, this compresses the tail of the distribution past r_{c} into a single bin. Again, protein pairs at the r_{c} value can be thought of as being very easily separable such that additional selectivity would not provide additional benefit in the separation. These distributions can now be thought of as the probability that a given resin set can separate a protein pair selected at random, where the area under the curve to the left of the red dotted line indicates the probability that the separation will fail.

Having defined a metric to calculate the distance distributions of resin sets, separability and orthogonality can be quantified. Separability can readily be quantified by calculating the distance distributions of protein retention time pairs (as described above) for a set of 1 to n resins. Orthogonality can then be calculated by quantifying the extent that the distance distribution has improved upon the addition of another resin. In an ideal system of two resins, for example, each resin would provide good separability, and when considered together provide synergistically enhanced separability, or orthogonality.

As discussed above, resins are described in a product-agnostic fashion by screening a large set of proteins on each resin and on combinations of those resins using gradient-elution chromatography processes. This large protein set samples a protein property space sufficiently such that the behavior of any additional protein that needs to be separated (product or impurity), is representable within this set.

While it is intuitive to think of the distance between two proteins, d_{ab} as discussed above, as their difference in elution salt concentration for one resin/condition combination, this definition can be modified when considering measuring such distances for pairs or triplets of resin/condition sets. In some embodiments, for systems using larger sets of resin/condition combinations, the distance, d_{ab}, in m resin/condition dimensions is defined as the maximal distance in any individual dimension. Thus, for a given set, M, containing m resin/condition combinations, the distance between proteins a and b can be calculated as:

=)

Without wishing to be bound by theory, utilizing this framework, it is possible to calculate a separability factor for any resin/condition combination or any set of resin/condition combinations. This framework enables the direct comparison of individual resin/condition combinations as well as sets of individual resin/condition combinations in terms of their ability to successfully separate pairs of proteins. It also facilitates the direct quantification of orthogonality for sets of individual resin/condition combinations on the basis of enhancement of individual or set separability. In this way, the embodiments of the present disclosure provide an approach for reducing high dimensional chromatographic data into quantitative separability and enhancement “scores.”

Referring again to FIG. 5, at 514, properties of the combination of M number of separation mediums and the library of components that increase orthogonality (E_{M}) of a set of separation mediums are identified. At 516, a target sample for separation is identified. At 518, a set of substantially orthogonal separation mediums for use in separating components of the target sample is selected. In some embodiments, the separability (S) of the set of substantially orthogonal separation mediums is greater than about 0.5. In some embodiments, the separability (S) of the set of substantially orthogonal separation mediums is greater than about 0.75. In some embodiments, the orthogonality (E_{M}) of the set of substantially orthogonal separation mediums is greater than about 0.2. In some embodiments, the orthogonality (E_{M}) of the set of substantially orthogonal separation mediums is greater than about 0.35. At 520, the target sample is administered to the set of substantially orthogonal separation mediums.

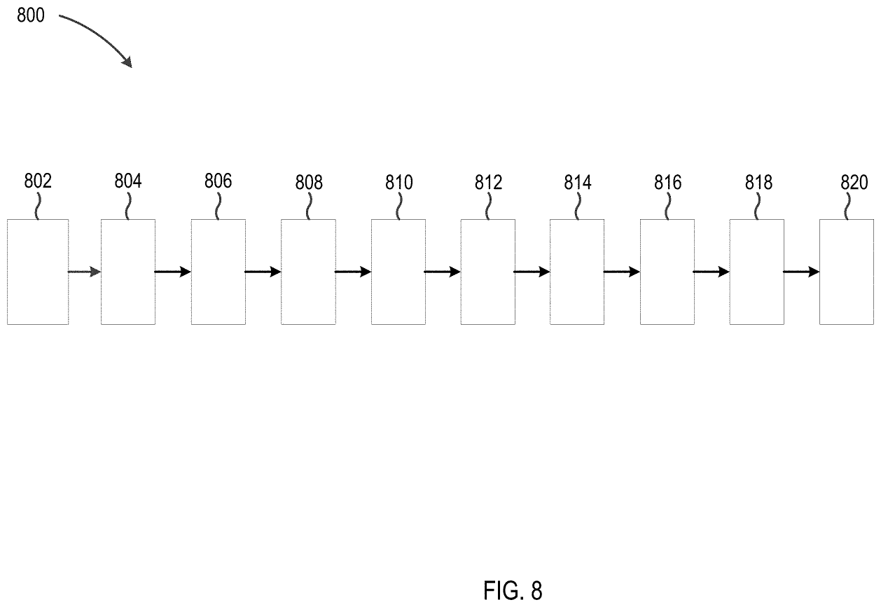

Referring now to FIG. 8, some embodiments of the present disclosure are directed to a method 800 for performing an optimized separation. At 802, a library of proteins for separation is prepared. At 804, a library of chromatographic separation resins is prepared. At 806, the library of proteins is administered to the chromatographic separation resins from the library of chromatographic separation resins and a plurality of combinations of the chromatographic separation resins, the chromatographic separation resins and the plurality of combinations of the chromatographic separation resins maintained at a first pH. At 808, the library of proteins is again administered to the chromatographic separation resins and the plurality of combinations of the chromatographic separation resins, however, this time the chromatographic separation resins and the plurality of combinations of the chromatographic separation resins are maintained at a different pH, e.g., at a second pH, subsequently at a third pH, etc. At 810, a separability (S) of the chromatographic separation resins and the plurality of combinations of the chromatographic separation resins is quantified, as discussed above. At 812, an orthogonality (E_{M}) of the combinations of chromatographic separation resins is quantified, also as discussed above. At 814, properties of the combinations of M number of chromatographic separation resins and the library of proteins that increase orthogonality (E_{M}) for a set of chromatographic separation resins are identified. At 816, a sample including two or more proteins is provided. At 818, a set of substantially orthogonal chromatographic separation resins for use in separating the two or more proteins based on the identified properties is selected. At 820, the sample is administered to the set of substantially orthogonal chromatographic separation resins. As discussed above, in some embodiments, the separability (S) of the set of M number of substantially orthogonal separation mediums is greater than about 0.5. In some embodiments, the separability (S) of the set of M number of substantially orthogonal separation mediums is greater than about 0.75. As discussed above, in some embodiments, the orthogonality (E_{M}) of the set of M number of substantially orthogonal separation mediums is greater than about 0.2. In some embodiments, the orthogonality (E_{M}) of the set of M number of substantially orthogonal separation mediums is greater than about 0.35.

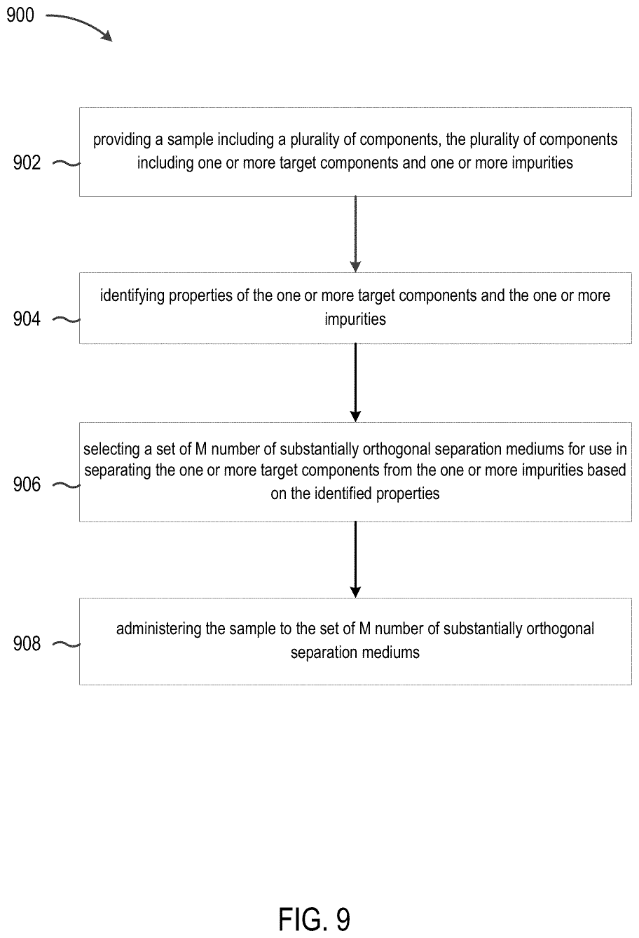

Referring now to FIG. 9, some embodiments of the present disclosure are directed to a method 900 for performing an optimized separation. At 902, a sample including a plurality of components is provided. In some embodiments, the plurality of components include one or more target components and one or more impurities. At 904, properties of the one or more target components and the one or more impurities are identified. At 906, a set of M number of substantially orthogonal separation mediums for use in separating the one or more target components from the one or more impurities based on the identified properties is selected. In some embodiments, a combination of substantially orthogonal separation mediums for the one or more target components and the one or more impurities based on the identified properties is designed. In some embodiments, the designed separation mediums or combinations of separation mediums are orthogonal or substantially orthogonal to themselves at different pH's. This allows a chromatographer to readily tune the selectivity between proteins by adjusting the pH, providing greater versatility for resin usage. At 908, the sample is administered to the set of M number of substantially orthogonal separation mediums. As discussed above, in some embodiments, the separability (S) of the set of M number of substantially orthogonal separation mediums is greater than about 0.5. In some embodiments, the separability (S) of the set of M number of substantially orthogonal separation mediums is greater than about 0.75. As discussed above, in some embodiments, the orthogonality (E_{M}) of the set of M number of substantially orthogonal separation mediums is greater than about 0.2. In some embodiments, the orthogonality (E_{M}) of the set of M number of substantially orthogonal separation mediums is greater than about 0.35.

### Methods

By way of example, a set of 15 model proteins was tested on a series of chromatographic separation resins ranging from strong to multimodal ion exchangers (both anionic and cationic) at various pH conditions. Without wishing to be bound by theory, the change in selectivity is highly dependent on the protein used as well as their titrations across the considered pH range, however, when considering the same set of proteins, one can readily identify resins which better interrogate selectivity differences caused by titrations of the considered protein set. Most resins studied have similar individual separability factors, while certain resin pairs at certain conditions were found to achieve high separability factors when paired, due to a high degree of orthogonality. Additionally, it was found that multimodal resins were consistently orthogonal to the other resins studied.

Sodium chloride, sodium phosphate monobasic, sodium phosphate dibasic, citric acid, trisodium citrate dihydrate, sodium hydroxide, Tris base, Chromasolv grade acetonitrile (ACN), sodium azide, Catalase from bovine liver, Trypsin, α-Lactalbumin from bovine milk, α-Chymotrypsinogen A (Type II) from bovine pancreas, Concanavalin A from Canavalia ensiformis, Lysozyme from chicken egg white, Cytochrome C from equine heart, α-Casein from bovine milk, α-Chymotrypsin (Type II) from bovine pancreas, Apoferritin from equine spleen, Lipoxidase from Glycine max (soybean), Ribonuclease B from bovine pancreas, Conalbumin from chicken egg white, Lipase (Type II) from porcine pancreas, Ubiquitin from bovine erythrocytes, Carbonic anhydrase from bovine erythrocytes, Hemoglobin (human), Lactoferrin from bovine milk, Phosphatase (alkaline) from bovine intestinal mucosa, β-Lactoglobulin B from bovine milk, α-Amylase (Type VIII-A) from barley malt, and album (rabbit) were purchase from Sigma-Aldrich (St. Louis, Mo.). Insulin Glargine was generously donated. hGH was generously donated by Novo Nordisk (Bagsværd, Denmark). Mab was generously donated by MedImmune (Gaithersburg, Md.). 96-well 350 μL sample collection plates, 96-well plate mats, Acquity UPLC Protein BEH C4 columns (300 angstrom, 1.7 μm, 2.1 mm×100 mm), and Acquity UPLC Protein BEH VanGuard Pre-Columns (300 angstrom, 1.7 μm, 2.1 mm×5 mm) were purchased from Waters Corporation (Milford, Mass.). Pre-packed OPUS® 200 MiniChrom columns (5 mm×10 mm) were purchased from Repligen (Waltham, Mass.) and packed with the following resins: Q Sepharose HP, SP Sepharose HP, Capto Adhere, Nuvia cPrime, Capto MMC, Capto MMC ImpRes, HyperCel STAR ΔX, HyperCel STAR CEX, CMM HyperCel, Eshmuno Q, Eshmuno HCX, BAKERBOND POLYQUAT, BAKERBOND POLYABx, and Toyopearl MX-Trp-650M. 350 μL, 0.2 μm Supor AcroPrep Advance filter plates were purchased from Pall Corporation (Port Washington, N.Y.). Mylar plate sealers and HPLC grade trifluoroacetic acid (TFA) were purchased from Thermo Fischer Scientific (Pittsburgh, Pa.). 0.2 μm centrifugal filters, 30 mL Luerlock syringes, 96-well 2 mL collection plates, and 0.2 μm PES syringe filters were purchased from VWR (Radnor, Pa.).

Model protein linear gradient screens were performed on an ÄKTA Explorer 10 system. Model proteins were grouped into 3 groups of 5 proteins. Each individual protein stock concentration was 2.0 mg/mL, which was measured by nanodrop after syringe filtering with a 0.2 μm PES filter. For each set, 5 proteins were added resulting in an individual protein concentration of 0.4 mg/mL for each protein, or a total protein concentration of 2 mg/mL for each mixture. A 1 mL injection of protein mixture was used for each run (using a P-960 sample pump at a flow rate of 1 CV/min), resulting in a total protein load challenge of −10 mg/mL for these 0.2 mL MiniChrom columns. Upon loading, a 5 CV re-equilibration step was performed followed by a 40 CV linear gradient from 0 to 1.5 M NaCl, followed by a 10 CV hold at 1.5 M NaCl. For resins with cation exchange functionality 10 CVs of 100 mM TRIS Base+1M NaCl was used as a first strip, whereas for resins with anion exchange functionality 10 CVs of 100 mM citric acid+1M NaCl was used as a first strip. The columns were then regenerated with 10 CV of 0.5 M NaOH, followed by re-equilibration. Flowthrough fractions were collected in volumes of 5 CVs. Fractions in the linear gradient, 1.5 M NaCl hold, and first strip (i.e. TRIS base/citric acid strips), were collected at a resolution of 1 CV. Fractions were not collected for the 0.5 M NaOH regeneration step. Collected fractions were analyzed using rapid UP-RPLC (either 3.4 minutes of 3.9 minutes depending on the set of model proteins considered) and were able to analytically separate all model proteins contained in each fraction. A Matlab® code was used to reconstruct the ÄKTA chromatogram peaks using the fractions analyzed by UP-RPLC and was used to perform curve smoothing using Matlab's “pchip” function which fits data to a piecewise cubic Hermite interpolating polynomial (1040 points were used when implementing the pchip function). This data was stored as a .mat file and all analysis codes were written in Matlab.

Referring now to Table 1 below, 25 model proteins were considered in these screens. Each protein was individually evaluated for their retention behavior on an UP-RPLC column (using a 10-minute linear acetonitrile gradient). It was found that many of these model proteins either exhibited poor solubility (at the selected conditions) or contained high levels of impurities such that resolution between the main product and its impurities was difficult to achieve, even by UP-RPLC. These model proteins were removed from consideration and only the 15 proteins shown in Table 1 were included in the ÄKTA chromatographic screens. The next step was to identify which proteins would be grouped together for the multi-protein injections on the ÄKTA screens. For grouping proteins, it was desired that proteins in a given group or set could easily be separated by analytical UP-RPLC such that their difference in retention times would be maximized. The retention times of the 15 selected model proteins and approximate peak widths from the individual protein UP-RPLC injections were recorded. A Matlab script was written which considered all permutations of 3 sets of 5 proteins from this list of 15 proteins. For a given permutation, the distance between each protein and its nearest neighbor protein were calculated.

Those selected for further screening included a first Set 1 proteins (1-5), second Set 2 proteins (6-10), and third Set 3 proteins (11-15).

TABLE 1
List of model proteins

| | Protein | pI | MW |
|---|---|---|---|
| 1 | Alpha-Lactalbumin | 5.0 | 14.1 |
| 2 | Alpha-Chymotrypsinogen A (Type II) | 8.5 | 25.7 |
| 3 | Horse Cytochrome C | 10.3 | 11.7 |
| 4 | hGH | 5.1 | 22 |
| 5 | mAb 2 | 8.3 | 150 |
| 6 | Concanavalin A, Type VI | 5 | 25.5 |
| 7 | Lysozyme | 11.4 | 14.3 |
| 8 | Alpha-Chymotrypsin | 9.2 | 25.2 |
| 9 | Ribonuclease B | 8.9 | 13.7 |
| 10 | Albumin (Rabbit) | 5.8 | 66.3 |
| 11 | Conalbumin | 6.7 | 75.8 |
| 12 | Ubiquitin | 6.8 | 8.6 |
| 13 | Carbonic Anhydrase | 6.4 | 29 |
| 14 | Lactoferrin | 8.7 | 82.4 |
| 15 | Beta-Lactoglobulin B | 5.1 | 18.2 |

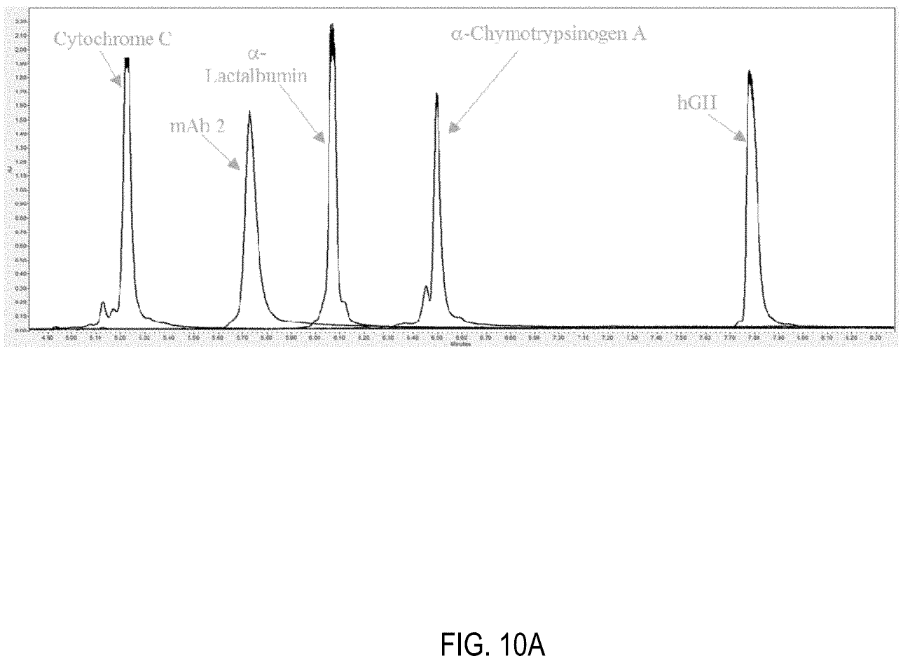

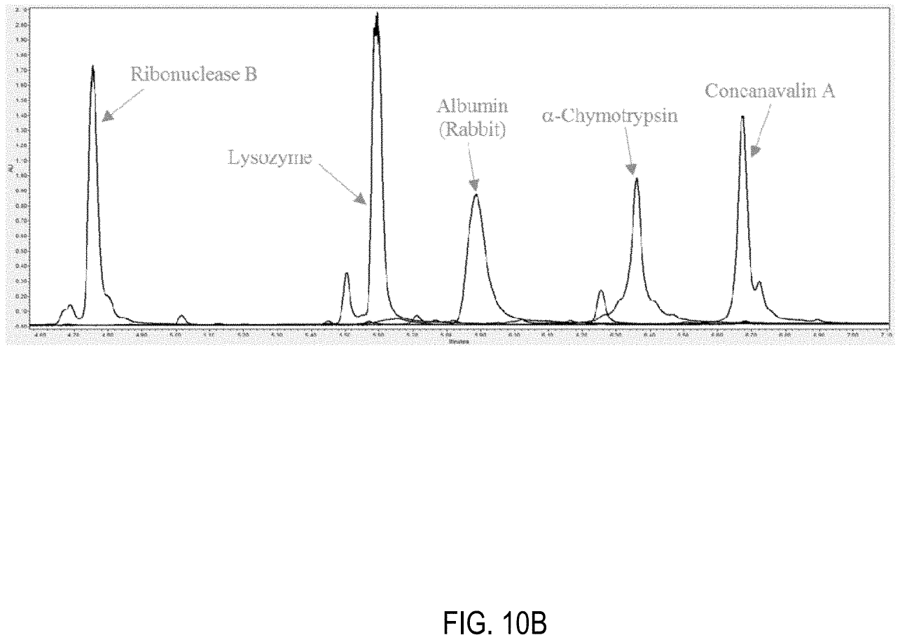

Referring to FIGS. 10A-10C, the overlaid UP-RPLC chromatograms (from the 10-minute linear acetonitrile gradient screens for individual proteins) of the model proteins in each of the three sets are shown. It can be seen that Set 1 (FIG. 10A) gives very good separation of its model proteins. Set 2 proteins (FIG. 10B) also show large retention time differences, however, it is worth noting that there are several impurity species present which elute nearby some of the main species. Finally, Set 3 proteins (FIG. 10C) exhibit good separation between most model proteins, except Conalbumin and Lactoferrin, which exhibit some peak overlap. Nevertheless, it was determined that the resolution would be sufficient and could be further improved during UP-RPLC methods development. After selecting the model proteins to group into sets, UP-RPLC methods development and optimization were performed in order to reduce each set's assay time as much as possible. After optimization, rapid assays were developed with sufficient peak separation and with run times of 3.9, 3.4, and 3.9 minutes for Set 1, 2, and 3 proteins, respectively.

Upon developing rapid UP-RPLC assays for the set of 15 model proteins, these proteins were screened in linear salt gradients from 0 to 1.5M NaCl at pH 5.0, 6.0, and 7.0 on a variety of resins with different functionalities. Table 2 lists the resins considered in this screen.

TABLE 2
List of resins and their chemical functionalities used for screening
product agnostic orthogonality on model proteins.

| Resin | Type |
|---|---|
| SP Sepharose HP | CEX |
| HyperCel STAR CEX | Salt Tolerant CEX |
| Capto MMC | MMC |
| Capto MMC ImpRes | MMC |
| Nuvia cPrime | MMC |
| CMM HyperCel | MMC |
| Toyopearl MX-Trp-650M | MMC |
| Eshmuno HCX | MMC |
| BakerBond Poly ABx | MMC/MMA |
| Q Sepharose HP | AEX |
| BakerBond PolyQuat | Tentacular AEX |
| Eshmuno Q | Tentacular AEX |
| HyperCel STAR AX | Salt Tolerant AEX |
| Capto Adhere | MMA |

CEX = Cation Exchanger,
MMC = Multimodal Cation Exchanger,
AEX = Anion Exchanger,
MMA = Multimodal Anion Exchanger.

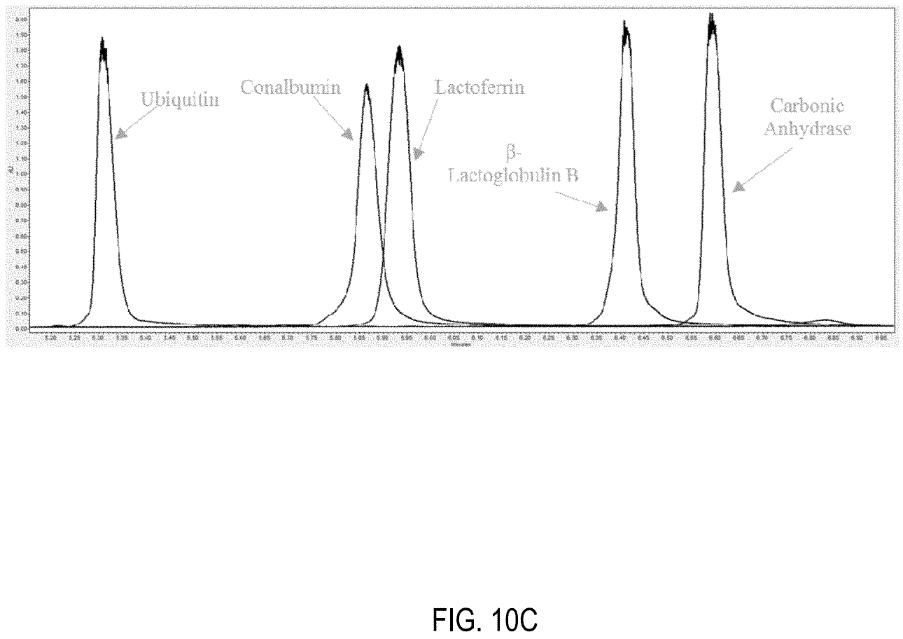

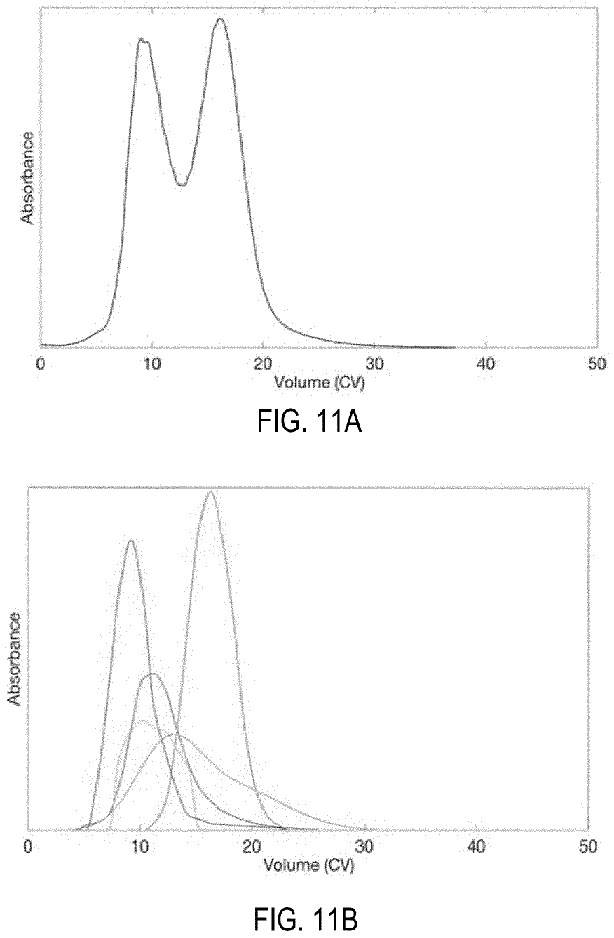

Screens were carried out using 5 proteins at a time (as determined by the proteins sets in FIG. 10A-10C and fractions (1 CV/each) were collected throughout each gradient. ÄKTA fractions containing a UV signal were subjected to UP-RPLC analysis and these analytical chromatograms were used to deconvolute the model protein UV signals in order to determine individual retention behaviors. FIGS. 11A-11B provide an example of this model protein deconvolution. FIG. 11A shows the ÄKTA chromatogram for Ribonuclease B, Lysozyme, Albumin (rabbit), α-Chymotrypsin, and Concanavalin A in a pH 5.0 linear salt gradient on Toyopearl MX-Trp-650M. From inspection of the ÄKTA chromatogram, only two distinct peaks can be discerned despite 5 proteins being injected, indicating that multiple species are likely present in each peak. Upon UP-RPLC fraction analysis and deconvolution, this was verified in FIG. 11B, where the convolution of all 5 model proteins is shown.

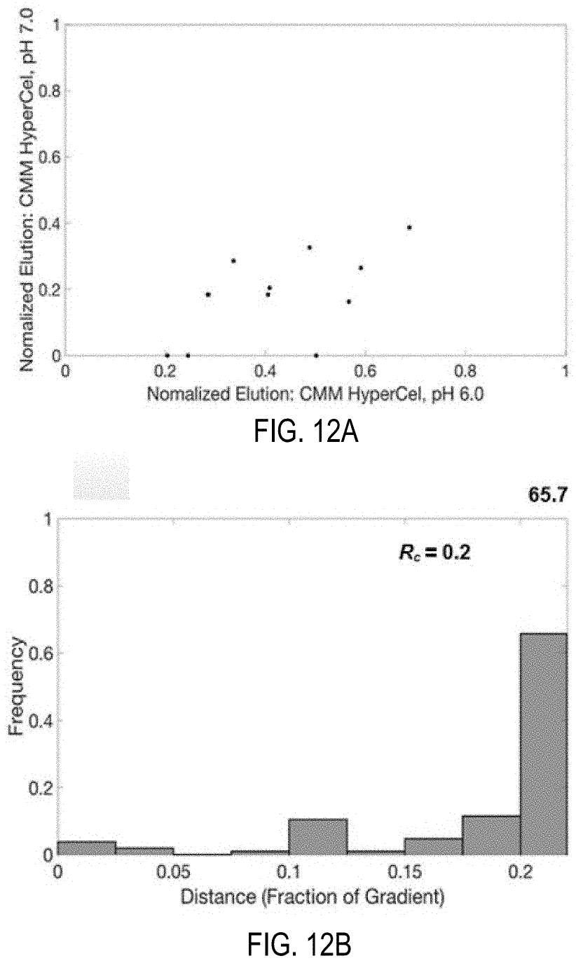

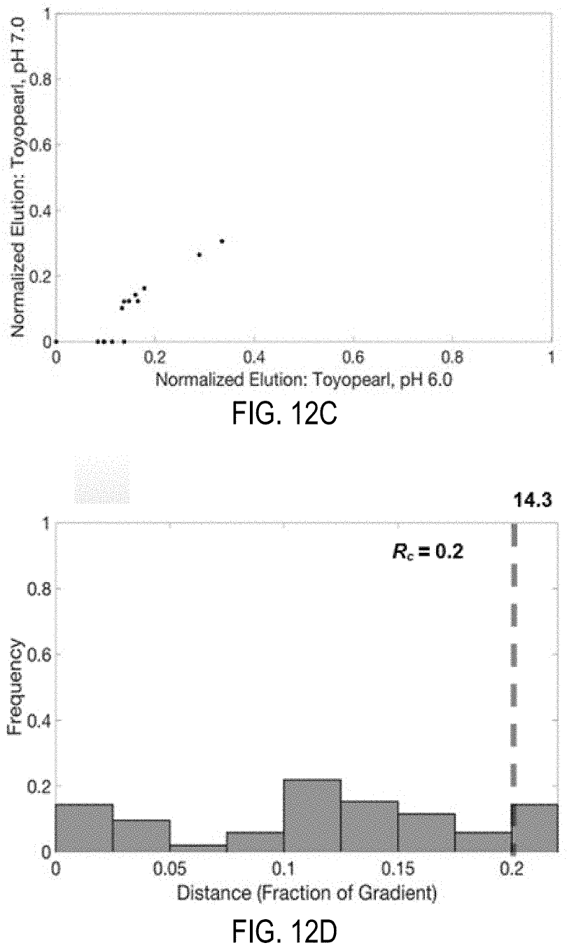

FIGS. 12A-12D show examples of two resins which exhibit very different orthogonal selectivity when operated at different pHs. FIG. 12A shows a scatterplot of CMM HyperCel operated at pH 6.0 (x-axis) and at pH 7.0 (y-axis). It can be visually seen from the scatterplot that this resin offers significant orthogonal selectivity between these two pH values due to the relatively large distance/spread between protein retention time points as well as the seemingly uncorrelated nature of the data, indicating selectivity order changes for this resin at different pHs. In order to quantify the extent of orthogonality between these two pH values, an r_{c} value of 0.2 and a failure region cutoff at 0.05 was used in embodiments of the methods described above, obtaining the distributions shown in FIGS. 12B and 12D. It can clearly be seen that a significant portion of the distribution is contained at r_{c} (˜65.7%), indicating significant separability. The separation value was calculated to be 0.847, meaning that the average distance is much closer to r_{c} than it is to the failure region, also supporting the claim that this set is highly separable. Orthogonality was calculated to be 0.202.

In contrast, Toyopearl MX-Trp-650M exhibited relatively poor separability and orthogonality to itself when compared at different pH values. FIG. 12C shows a model protein retention scatterplot for Toyopearl MX-Trp-650M at pH 6.0 (x-axis) vs. pH 7.0 (y-axis). It can clearly be seen from the scatterplot that the points are more clustered and that a visual linear correlation appears to exist between these points. Again, in order to quantify the extent of orthogonality between the two pHs (FIG. 12D). This distance distribution is clearly quite different than the one shown in FIG. 12B. Firstly, the percent of the distribution that is at r_{c} is much lower (14.3%) than that for CMM HyperCel. Also, the separability value calculated for the Toyopearl pH pair is 0.495, which is smaller than that for CMM HyperCel. Finally, the orthogonality value calculated for this Toyopearl pH pair is 0.137, indicating less orthogonality (or enhancement) than for the case with CMM HyperCel. Both of these metrics indicate that CMM HyperCel is more versatile in that it is able to drastically change its selectivity at different pHs.

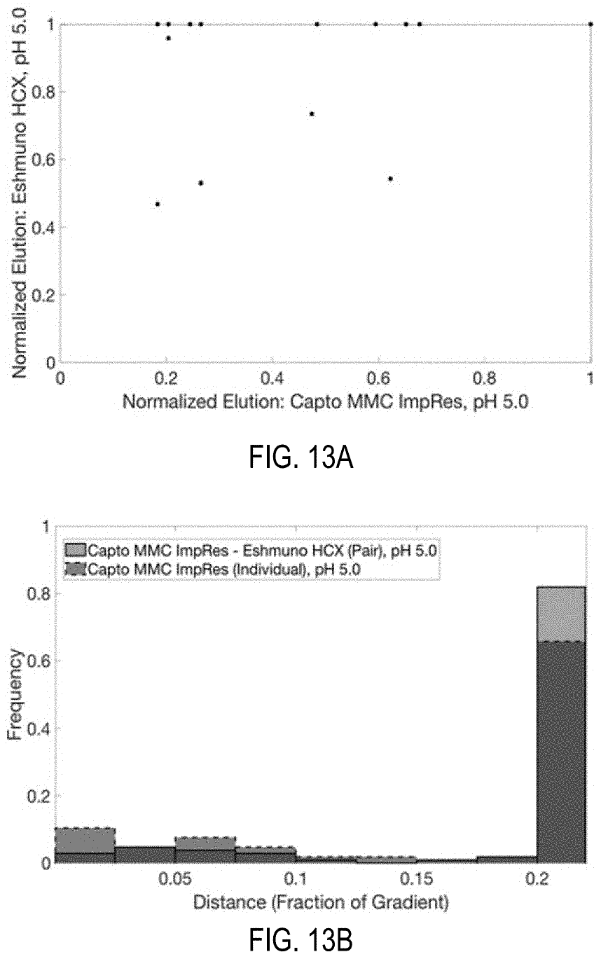

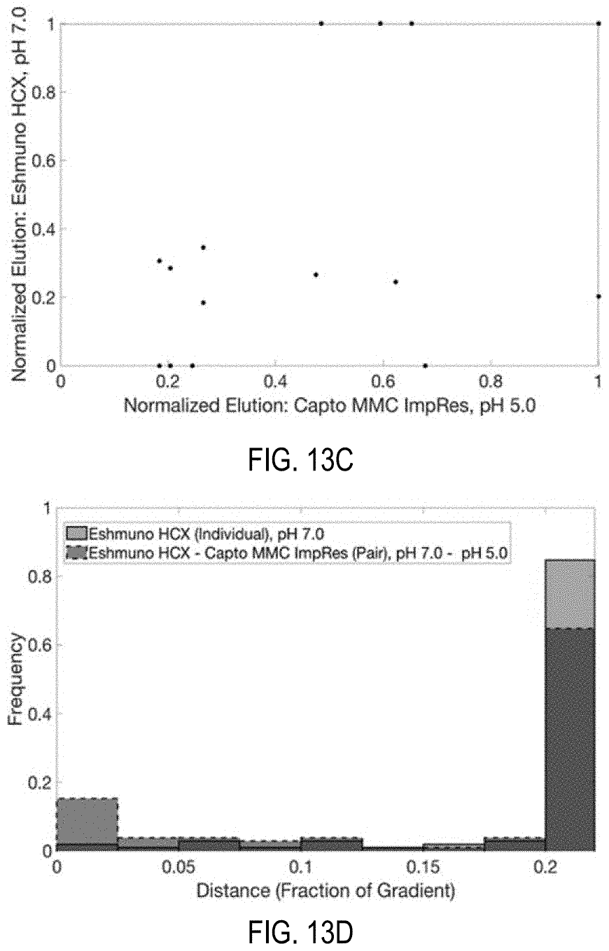

The extent to which multimodal resins within the same family are orthogonal to each other was also investigated. Though the focus of this investigation was on multimodal cation exchangers, this analysis can easily be performed on multimodal anion exchangers. As discussed elsewhere herein, an ideal pair of resins will exhibit good individual separability and offer improved synergistic separability (or orthogonality) when operated together. Referring now to FIGS. 13A-13D, a pair of multimodal cation exchangers which exhibit a high degree of orthogonality as determined by percent contained at r_{c}. FIGS. 13A-13B show Capto MMC ImpRes and Eshmuno HCX both at pH 5.0 and FIGS. 13C-13D show Capto MMC ImpRes and Eshmuno HCX at pH 5.0 and 7.0, respectively. FIGS. 13A and 13C show scatterplots for the model protein retention times. FIG. 13B shows an overlaid histogram of the Capto MMC ImpRes (pH 5.0) 1D distance distribution and the Capto MMC ImpRes-Eshmuno HCX pair distance distribution. FIG. 13D shows an overlaid histogram of the Eshmuno HCX (pH 7.0) 1D distance distribution and the Capto MMC ImpRes-Eshmuno HCX pair distance distribution. K=0.2 and the failure region cutoff was 0.05. Separation values for Capto MMC ImpRes and Eshmuno HCX both operated at pH 5.0 were found to be 0.717 and 0.449, respectively. The separation value calculated for this set was 0.857. The orthogonality value calculated for this set was 0.196. This can visually be seen in the overlaid histogram in FIG. 13B. It can clearly be seen that there is a lower frequency in the short distance region when these resins are considered together. In addition, a significant improvement in the frequency at r_{c} is observed. The constraint that resins be operated at the same pH was then relaxed in FIGS. 13C and 13D. The Capto MMC ImpRes and Eshmuno HCX, this time operated at pH 5.0 and 7.0, had calculated separation values of 0.717 and 0.718, respectively. As with the previous case, the calculated separation value of the combination (0.910) is higher than the individual values, resulting in a calculated orthogonality value of 0.266. Without wishing to be bound by theory, Capto MMC ImpRes is a small molecule ligand with an agarose base matrix whereas Eshmuno HCX is a tentacular ligand with a polyvinyl ether base matrix. These different chemistries and ligand presentations likely contribute to the high orthogonality.

Methods and systems of the present disclosure utilize orthogonality concepts used for analytical chromatography and apply them to chromatography for downstream processing applications. Herein is developed a framework for constraining the design space based on identifying a small set of product-agnostic, optimally orthogonal resins with which most separations (capture or polishing) can be accomplished. These methods and systems are advantageous to robustly calculate orthogonality for any number of resins in an arbitrarily large, defined system of proteins without the overestimation and underestimation biases previously discussed. By performing separations with two or three optimally orthogonal resins, impurity removal can be maximized with a minimal number of steps.

This approach operates in a product-agnostic manner such that it is able to identify universal orthogonality in separation systems. In addition, it provides a robust strategy for quantifying separability and orthogonality in separation systems regardless of the system complexity, e.g. number of proteins, number of resins, number of conditions tested, and type of data, e.g., direct chromatographic retention data, adsorption isotherm data, etc. By identifying these product-agnostic, optimally orthogonal resins before the process development timeline, it is possible to significantly constrain the design space and speed up process development. Ultimately, such an approach will lead to 1) faster process development and 2) more cost-effective processes (for non-affinity processes).

This approach for determining product agnostic orthogonality can be used for process development and resin selection. Additionally, this approach can be used as an integral tool for resin development in order to create optimally orthogonal multimodal resins. This framework and software tool has important industrial applications in the biomanufacturing space. For example, it can improve the efficiency in downstream process development by guiding resin selection and expediting high-throughput screens. It can also be an invaluable tool for chromatographic resin manufacturers by providing an easy-to-use tool to evaluate the efficacy of new chromatographic materials, including next-generation multimodal resins.

Although the invention has been described and illustrated with respect to exemplary embodiments thereof, it should be understood by those skilled in the art that the foregoing and various other changes, omissions and additions may be made therein and thereto, without parting from the spirit and scope of the present invention.

1. A method for determining an optimized separation comprising:
preparing a library of components for separation;
preparing a library of separation mediums;
administering the library of components to the separation mediums from the library of separation mediums and a plurality of combinations of the separation mediums to separate pairs of components in the library of components;
quantifying a separability (S) of the separation mediums and the combinations of separation mediums, wherein the separability (S) is a measure of a probability that the separation medium or combination of separation mediums will separate a pair of components in the library of components; and
quantifying an orthogonality (E_{M}) of the combinations of separation mediums, wherein the orthogonality (E_{M}) is a measure of enhancement in separability (S) of a combination of M number of separation mediums over M−1 number of separation mediums.

2. The method according to claim 1, wherein administering the library of components further comprises:
administering the library of components to the separation mediums from the library of separation mediums and the plurality of combinations of separation mediums at varying pH.

3. The method according to claim 1, further comprising:
identifying properties of the combination of M number of separation mediums and the library of components that increase orthogonality (E_{M}) of a set of separation mediums;
identifying a target sample for separation; and
selecting a set of substantially orthogonal separation mediums for use in separating components of the target sample.

4. The method according to claim 3, further comprising:
administering the target sample to the set of substantially orthogonal separation mediums.

5. The method according to claim 3, wherein the separability (S) of the set of substantially orthogonal separation mediums is greater than about 0.5.

6. The method according to claim 5, wherein the separability (S) of the set of substantially orthogonal separation mediums is greater than about 0.75.

7. The method according to claim 3, wherein the orthogonality (E_{M}) of the set of substantially orthogonal separation mediums is greater than about 0.2.

8. The method according to claim 7, wherein the orthogonality (E_{M}) of the set of substantially orthogonal separation mediums is greater than about 0.35.

9. The method according to claim 1, wherein the separability (S) is calculated according to the following Formula I:
$$S = \frac{1}{( \begin{matrix}n \\ 2\end{matrix} )} {\sum }_{a = 1}^{n - 1} {\sum }_{b = a + 1}^{n} {w}_{ab} \qquad ( Formula I )$$
wherein
$${w}_{ab} = \{ \begin{matrix}0 , & {d}_{ab} < {r}_{low} \\ \frac{{d}_{ab} - {r}_{low}}{{r}_{high} - {r}_{low}} , & {r}_{high} > {d}_{ab} > {r}_{low} \\ 1 , & {d}_{ab} > {r}_{high}\end{matrix} \} ,$$
and
wherein n is the number of components in the library, d_{ab} is the separation distance between a first component (a) and a second component (b) on a separation medium, r_{high} represents the threshold above which first component (a) and second component (b) are considered successfully separated and now represents the threshold below which first component (a) and second component (b) are considered unsuccessfully separated.

10. The method according to claim 1, wherein the orthogonality (E_{M}) is calculated according to the following Formula II:
$${E}_{M} = \frac{{S}_{M}}{max ( {S}_{M - 1} ∀ resin / condition combinations in M )} - 1 \qquad ( Formula II )$$

11. A method for performing an optimized separation, comprising:
providing a sample including a plurality of components, the plurality of components including one or more target components and one or more impurities;
identifying properties of the one or more target components and the one or more impurities;
selecting a set of M number of substantially orthogonal separation mediums for use in separating the one or more target components from the one or more impurities based on the identified properties, wherein an orthogonality (E_{M}) of the set of substantially orthogonal separation mediums is a measure of enhancement in separability (S) of the M number of separation mediums over M−1 number of separation mediums; and
administering the sample to the set of M number of substantially orthogonal separation mediums,
wherein the separability (S) is calculated according to the following Formula I:
$$S = \frac{1}{( \begin{matrix}n \\ 2\end{matrix} )} {\sum }_{a = 1}^{n - 1} {\sum }_{b = a + 1}^{n} {w}_{ab} \qquad ( Formula I )$$
wherein
$${w}_{ab} = \{ \begin{matrix}0 , & {d}_{ab} < {r}_{low} \\ \frac{{d}_{ab} - {r}_{low}}{{r}_{high} - {r}_{low}} , & {r}_{high} > {d}_{ab} > {r}_{low} \\ 1 , & {d}_{ab} > {r}_{high}\end{matrix} \} ,$$
and
wherein n is the number of components, d_{ab} is the separation distance between a first component (a) and a second component (b) on a separation medium, r_{high} represents the threshold above which first component (a) and second component (b) are considered successfully separated, and r_{low} represents the threshold below which first component (a) and second component (b) are considered unsuccessfully separated.

12. The method according to claim 11, wherein the separability (S) of the set of M number of substantially orthogonal separation mediums is greater than about 0.5.

13. The method according to claim 12, wherein the separability (S) of the set of M number of substantially orthogonal separation mediums is greater than about 0.75.

14. The method according to claim 11, wherein the orthogonality (E_{M}) of the set of M number of substantially orthogonal separation mediums is greater than about 0.2.

15. The method according to claim 14, wherein the orthogonality (E_{M}) of the set of M number of substantially orthogonal separation mediums is greater than about 0.35.

16. The method according to claim 11, wherein the orthogonality (E_{M}) is calculated according to the following Formula II:
$${E}_{M} = \frac{{S}_{M}}{max ( {S}_{M - 1} ∀ resin / condition combinations in M )} - 1 \qquad ( Formula II )$$

17. The method according to claim 11, wherein selecting the set of M number of substantially orthogonal separation mediums further comprises:
designing a combination of substantially orthogonal separation mediums for the one or more target components and the one or more impurities based on the identified properties.

18. A method for performing an optimized separation comprising:
preparing a library of proteins for separation;
preparing a library of chromatographic separation resins;
first administering the library of proteins to the chromatographic separation resins from the library of chromatographic separation resins and a plurality of combinations of the chromatographic separation resins, wherein the chromatographic separation resins and the plurality of combinations of the chromatographic separation resins are maintained at a first pH;
subsequently administering the library of proteins to the chromatographic separation resins and the plurality of combinations of the chromatographic separation resins, wherein the chromatographic separation resins and the plurality of combinations of the chromatographic separation resins are maintained at at least a second pH;
quantifying a separability (S) of the chromatographic separation resins and the plurality of combinations of the chromatographic separation resins, wherein the separability (S) is a measure of a probability that the chromatographic separation resin or the combination of chromatographic separation resins will separate a pair of proteins in the library of proteins;
quantifying an orthogonality (E_{M}) of the combinations of chromatographic separation resins, wherein the orthogonality (E_{M}) is a measure of the enhancement in separability (S) of a combination of M number of chromatographic separation resins over M−1 number of chromatographic separation resins;
identifying properties of the combinations of M number of chromatographic separation resins and the library of proteins that increase orthogonality (E_{M}) for a set of chromatographic separation resins;
providing a sample including two or more proteins;
selecting a set of substantially orthogonal chromatographic separation resins for use in separating the two or more proteins based on the identified properties; and
administering the sample to the set of substantially orthogonal chromatographic separation resins.

19. The method according to claim 18, wherein the separability (S) is calculated according to the following Formula I:
$$S = \frac{1}{( \begin{matrix}n \\ 2\end{matrix} )} {\sum }_{a = 1}^{n - 1} {\sum }_{b = a + 1}^{n} {w}_{ab} \qquad ( Formula I )$$
wherein
$${w}_{ab} = \{ \begin{matrix}0 , & {d}_{ab} < {r}_{low} \\ \frac{{d}_{ab} - {r}_{low}}{{r}_{high} - {r}_{low}} , & {r}_{high} > {d}_{ab} > {r}_{low} \\ 1 , & {d}_{ab} > {r}_{high}\end{matrix} \} ,$$
and
wherein n is the number of proteins in the library, d_{ab} is the separation distance between a first protein (a) and a second protein (b) on a separation medium, No represents the threshold above which first protein (a) and second protein (b) are considered successfully separated and r_{low} represents the threshold below which first protein (a) and second protein (b) are considered unsuccessfully separated, and
wherein the orthogonality (E_{M}) is calculated according to the following Formula II:
$${E}_{M} = \frac{{S}_{M}}{max ( {S}_{M - 1} ∀ resin / condition combinations in M )} - 1. \qquad ( Formula II )$$

20. The method according to claim 19, wherein the separability (S) of the set of substantially orthogonal separation mediums is greater than about 0.75, and the orthogonality (E_{M}) of the set of substantially orthogonal separation mediums is greater than about 0.35.
